(19) United States
(12) Patent Application Publication
(10) Pub. No.: US 2013/0274615 A1
(43) Pub. Date: Oct. 17, 2013

(54) Measurement of Cerebral Physiologic Parameters Using Bioimpedance

(71) Applicants: Shlomi Ben-Ari, Benyamina (IL); Shmuel Marcovitch, Kefar-Saba (IL)

(72) Inventors: Shlomi Ben-Ari, Benyamina (IL); Shmuel Marcovitch, Kefar-Saba (IL)

(21) Appl. No.: 13/861,521

(22) Filed: Apr. 12, 2013

Related U.S. Application Data
(60) Provisional application No. 61/623,206, filed on Apr. 12, 2012.

(51) Int. Cl. A61B 5/0205; A61B 5/053

(52) U.S. Cl. CPC A61B 5/0205; A61B 5/0535
USPC 600/483; 600/547

(57) ABSTRACT

Devices and methods for monitoring intracranial physiological parameters, including intracranial pressure, cerebral perfusion pressure, cerebral blood flow, cerebral blood volume, edema status, and brain compliance are disclosed. In one aspect, an apparatus may involve receiving at least one impedance plethysmography signal. Waveforms may be extracted from the impedance plethysmography signals and used for estimating the intracranial physiological parameters. Various characteristics may be determined from the waveforms to aid in the estimation of intracranial physiological parameters.

### RELATED APPLICATIONS

This application claims the benefit of priority under 35 U.S.C. §119(e) of U.S. Provisional Application No. 61/623,206, filed Apr. 12, 2012, the entirety of which is incorporated herein by reference.

### TECHNICAL FIELD

The instant disclosure describes, among other things, mechanisms for detecting and/or monitoring cerebral pathologies.

### BACKGROUND

Cerebral pathologies may lead to temporary brain damage injury, permanent brain damage injury, or death. Examples of cerebral pathologies include strokes, trauma, edema and traumatic brain injury (TBI). Symptoms of these cerebral pathologies often include increased intracranial pressure (ICP). When brain tissue is injured, for example, the injured tissue may develop edema and hemorrhage, both resulting in an increased ICP. To prevent additional brain damage, one practice may include monitoring the ICP by insertion of a pressure probe into the cranial space. This is an invasive procedure typically involving drilling through the skull (usually at an un-affected area), inserting the probe through the drilled hole, and securing the probe with a nut to the skull or by tunneling a catheter through the scalp. This invasive method typically involves risks associated with insertion of a probe into healthy brain tissue or the ventricular space and risks of infection by an invasive probe.

A non-invasive method and apparatus may be used to measure and monitor ICP and additional intracranial physiological parameters that may be clinically useful for diagnosing strokes, trauma, and other conditions that can affect the functioning of the brain. These parameters may include, for example, cerebral blood volume, cerebral blood flow, cerebral perfusion pressure, vascular autoregulation functioning and cerebral edema status.

One way to monitor or detect ICP and additional intracranial physiological parameters may include physically inserting a probe into the cerebrospinal fluid or into an artery, angiography, computed tomography angiography (CTA), perfusion computed tomography (PCT), transcranial doppler ultrasound (TCD), positron emission tomography (PET), and magnetic resonance imaging (MRI) and angiography (MRA). Some non-invasive methods for detecting or monitoring ICP and additional intracranial physiological parameters may require, for example, machines for carrying out CT, PCT, PET, and/or MRI procedures. In some instances, the lack of continuous monitoring, the cost of these machines, their limited mobility, and/or their significant expense per use, may limit their usefulness in situations where either regular, continuous, or frequent monitoring of intracranial physiological characteristics may be desirable.

The foregoing description is merely exemplary for providing general background and is not restrictive of the various embodiments of systems, methods, devices, and features as described and claimed.

### SUMMARY OF A FEW ASPECTS OF THE DISCLOSURE

In the presently disclosed embodiments, several exemplary methods and systems are described that may be used to estimate ICP and additional intracranial physiological parameters. In some embodiments, these methods and systems may be useful, for example, for continuous or frequent use and may involve, for example, electrodes, and/or a patient headset and cerebral perfusion monitor for acquiring impedance signals and extracting waveforms for estimating ICP and additional intracranial physiological parameters.

One exemplary disclosed embodiment includes an intracranial physiological measurement apparatus. An intracranial physiological measurement apparatus may include at least one processor. The at least one processor may be configured to receive at least one impedance plethysmography signal associated with a brain of a subject, extract at least one impedance plethysmography characteristic from the impedance plethysmography signal, and estimate mean intracranial pressure from the at least one impedance plethysmography characteristic.

Another exemplary embodiment includes an intracranial physiological measurement apparatus. An intracranial physiological measurement apparatus according to this embodiment may include at least one processor configured to receive at least one impedance plethysmography signal associated with a brain of a subject, extract at least one impedance waveform associated with a physiological process from the impedance plethysmography signal, and estimate a working position on a brain compliance curve based on the at least one impedance waveform associated with a physiological process.

Another exemplary embodiment includes an intracranial physiological measurement apparatus. An intracranial physiological measurement apparatus according to this embodiment may include at least one processor configured to transmit and receive a plurality of impedance measurement signals at a plurality of frequencies to at least one pair of electrodes, generate a plurality of impedance measurements of a head of a subject at the plurality of frequencies, and estimate a physiologic parameter of a brain of the subject based on the plurality of impedance measurements.

### BRIEF DESCRIPTION OF THE DRAWINGS

The accompanying drawings, which are incorporated in and constitute a part of this specification, together with the description, serve to explain the principles of the embodiments described herein.

FIG. 1 provides a diagrammatic representation of an exemplary intracranial physiological measurement apparatus consistent with exemplary embodiments of the invention;

FIG. 2 provides a diagrammatic representation of major cerebral arteries;

FIG. 3 provides a diagrammatic representation of exemplary bioimpedance signal pathways in the brain of a subject consistent with exemplary embodiments of the invention;

FIG. 4a provides a diagrammatic representation of an intracranial pressure waveform obtained from a healthy brain under normal conditions;

FIG. 4b provides a diagrammatic representation of an intracranial pressure waveform obtained from a pathological brain;

FIG. 4c provides a diagrammatic representation of an intracranial pressure waveform obtained from a brain under elevated intracranial pressure conditions;

FIG. 5a provides a diagrammatic representation of an exemplary intracranial pressure waveform;

FIG. 5b provides a diagrammatic representation of an exemplary impedance magnitude waveform, recorded simultaneously to the intracranial pressure waveform, consistent with embodiments of the invention;

FIG. 5c provides a diagrammatic representation of an exemplary impedance phase waveform, recorded simultaneously to the intracranial pressure waveform, consistent with embodiments of the invention;

FIG. 6 illustrates an intracranial pressure waveform of a brain with a high level of edema, or fluid buildup;

FIG. 7 diagrammatically illustrates a brain compliance curve;

FIG. 8 is a graph illustrating diastolic values of intracranial pressure and arterial blood pressure during respiratory cycles; and

FIG. 9 illustrates an exemplary tissue bioimpedance model.

### DETAILED DESCRIPTION

Reference will now be made in detail to exemplary embodiments as with reference to the accompanying drawings. In some instances, the same reference numbers will be used throughout the drawings and the following description to refer to the same or like parts. These embodiments are described in sufficient detail to enable those skilled in the art to practice the invention and it is to be understood that other embodiments may be utilized and that changes may be made without departing from the scope of the present invention. The following detailed description, therefore, is not to be interpreted in a limiting sense.

Unless otherwise defined, all technical and/or scientific terms used herein have the same meaning as commonly understood by one of ordinary skill in the art to which the embodiments of the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Exemplary disclosed embodiments may include devices and methods for the reception and analysis of impedance plethysmography (IPG) signals representing bioimpedance. More specifically, they may include apparatuses for receiving and analyzing signals and outputting information for estimating physiological brain conditions. In some embodiments consistent with the present disclosure, the estimated physiological brain conditions may include conditions associated with ICP. In some embodiments, the estimated physiological brain conditions may be conditions associated with a mean value of ICP.

As used herein, the term “mean value of ICP” refers to the average level of intracranial pressure as measured over a time interval of longer than a heartbeat. In some embodiments, the mean value of ICP refers to the average level of intracranial pressure as measured over a time interval corresponding to an integer number of heartbeats, such that pulsatile or dynamic components are averaged out. The time value over which a mean value of ICP is measured may be as short as a single heartbeat, or may stretch over many minutes or hours. The mean value of the ICP may, in fact, be dynamic itself. Due to such factors as edema development, fluid accumulation, and patient consciousness, the mean value of ICP as measured over, for example, one minute, may vary over the course of hours or days. These changes in the mean value of ICP may be characterized by time scales ranging from approximately half an hour to hours or days.

ICP may be determined based on three factors, including cerebral blood volume (CBV), which is affected by cerebral blood flow, edema status (i.e. fluid buildup), and cerebral spinal fluid (CSF) volume. Thus, in some embodiments, ICP may be estimated and monitored through determining CBV, edema status, and/or CSF volume. Exemplary devices and methods disclosed herein describe means of monitoring, estimating, and determining CBV, edema status, and CSF volume through the usage of IPG.

Impedance plethysmography (IPG), may be used to measure ICP. In IPG measurement of ICP, electrodes placed externally on the scalp, neck, and/or chest may be used to drive current into the patient and measure the induced voltage. An impedance plethysmography (IPG) measurement apparatus may be used to measure two sets of induced voltages associated with the right and left hemispheres of the patient or different sections.

Embodiments consistent with the present disclosure may include an IPG measurement apparatus. An IPG measurement apparatus may include (but does not necessarily include), for example, support elements such as a headset, headband, or other framework elements to carry or house additional functional elements. Further structures that may be incorporated may include electrodes, circuitry, processors, sensors, wires, transmitters, receivers, and other devices suitable for obtaining, processing, transmitting, receiving, and analyzing electrical signals. An intracranial physiological measurement apparatus may additionally include fasteners, adhesives, and other elements to facilitate attachment to a subject's body. As used herein, an intracranial physiological measurement apparatus need not include all such features.

Embodiments consistent with the present disclosure may include a measurement apparatus for non-invasive intracranial physiological parameters. An intracranial physiological measurement apparatus may include (but does not necessarily include), for example, support elements such as a headset, headband, or other framework elements to carry or house additional functional elements. Further structures that may be incorporated may include electrodes, circuitry, processors, sensors, wires, transmitters, receivers, and other devices suitable for obtaining, processing, transmitting, receiving, and analyzing electrical signals. An intracranial physiological measurement apparatus may additionally include fasteners, adhesives, and other elements to facilitate attachment to a subject's body. As used herein, an intracranial physiological measurement apparatus need not include all such features.

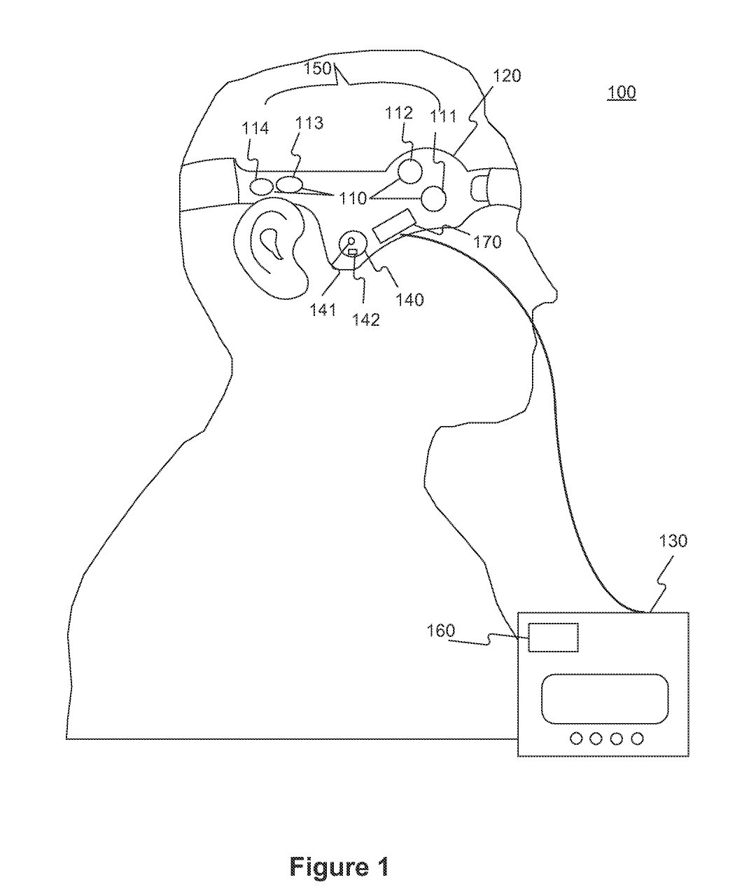

FIG. 1 provides a diagrammatic representation of an exemplary intracranial physiological measurement apparatus 100. This exemplary apparatus 100 may include electrodes 110 affixed to a subject's head via a headset 120. Electrodes 110 may be connected to cerebral perfusion monitor 130 via wires (or may alternatively include a wireless connection).

In some exemplary embodiments consistent with the disclosure, an intracranial physiological measurement apparatus may include at least one processor configured to perform an action. As used herein, the term “processor” may include an electric circuit that performs a logic operation on an input or inputs. For example, such a processor may include one or more integrated circuits, microchips, microcontrollers, microprocessors, all or part of a central processing unit (CPU), graphics processing unit (GPU), digital signal processors (DSP), field-programmable gate array (FPGA) or other circuit suitable for executing instructions or performing logic operations. The at least one processor may be configured to perform an action if it is provided with access to, is programmed with, includes, or is otherwise made capable carrying out instructions for performing the action. The at least one processor may be provided with such instructions either directly through information permanently or temporarily maintained in the processor, or through instructions accessed by or provided to the processor. Instructions provided to the processor may be provided in the form of a computer program comprising instructions tangibly embodied on an information carrier, e.g., in a machine-readable storage device, or any tangible computer-readable medium. A computer program may be written in any form of programming language, including compiled or interpreted languages, and it can be deployed in any form, including as a standalone program or as one or more modules, components, subroutines, or other unit suitable for use in a computing environment. The at least one processor may include specialized hardware, general hardware, or a combination of both to execute related instructions. The processor may also include an integrated communications interface, or a communications interface may be included separate and apart from the processor. The at least one processor may be configured to perform a specified function through a connection to a memory location or storage device in which instructions to perform that function are stored.

Consistent with some embodiments of the invention, the at least one processor may be configured to receive a signal. As used herein, a signal may include any time-varying or spatially-varying quantity. Receiving a signal may include obtaining a signal through conductive means, such as wires or circuitry; reception of a wirelessly transmitted signal; and/or reception of a signal previously recorded, such as a signal stored in memory. Receiving a signal may further encompass other methods known in the art for signal reception.

At least one processor 160, schematically illustrated in FIG. 1, configured to receive and analyze one or more IPG signals associated with a brain of a subject, may be included in Cerebral Perfusion Monitor 130, as part of exemplary intracranial physiological measurement apparatus 100. Processor 160 may be configured to perform all or some of the signal analysis methods described herein, or some of those functions may be performed by a separate processor. Processor 160 may also be configured to perform any common signal processing task known to those of skill in the art, such as filtering, noise-removal, etc. Processor 160 may further be configured to perform pre-processing tasks specific to the signal analysis techniques described herein. Such pre-processing tasks may include, but are not limited to, removal of signal artifacts, such as motion artifacts.

An IPG signal may represent bioimpedance information of a subject. When recorded from electrodes attached to the head of a subject, an IPG signal may be associated with the brain of the subject and may represent bioimpedance information of the subject's brain tissue. An IPG signal may also contain information about the electrical impedance of the subject between any two portions of a subject's body, depending on the placement of suitable electrodes. Information about the electrical impedance of the subject may include information about the resistive and/or reactive components of electrical impedance. According to the present disclosure, in some exemplary embodiments an IPG signal may be measured as a response signal to at least one measurement voltage signal, and/or at least one measurement current signal. An IPG signal, as used herein, may include one or more of the response signal and the measurement signal. According to the present disclosure, an IPG signal may be obtained discontinuously or substantially constantly from a subject. Even when data is obtained continuously in an analog fashion it may be obtained at a fixed or variable digital sampling rate high enough to capture characteristics of interest within the signal. As used herein, a constantly obtained signal refers to a signal obtained substantially constantly. A constantly obtained signal may contain discontinuities, at either regular or irregular intervals, but also contains enough data to generate a temporal reconstruction of characteristics of interest within the signal. For example, a constantly obtained IPG signal may be acquired using a digital sampling rate of 20 MSamples/sec (MS/sec) over a period of several minutes or hours. A sampling rate of 20 MS/sec may be sufficient to capture any voltage/current signals generated in the frequency range of 1 KHz-1 MHz. After obtaining the IPG signal by demodulating the voltage measurement with respect to the current measurement, it may be decimated to a lower sampling rate of, for example, 625 S/sec which is sufficient to capture any waveform characteristics that may be associated with a cardiac cycle of the subject, having time scales in hundredths of seconds. Characteristics of interest that may be captured in data extracted from a constantly obtained IPG signal will be discussed in further detail below.

According to the present disclosure, one or more waveforms may be extracted from an IPG signal. Extracted waveforms may include, for example, waveforms representative of impedance components and their change over time. Impedance components, may include, for example, the magnitude and phase of the impedance, or the resistive and reactive components of the impedance. Extracted waveforms may also be characterized by various combinations of these components. As used herein, a waveform may be considered “extracted” from an IPG signal if it may be derived from the IPG signal or if it may be determined using the IPG signal.

By way of example only, extracted waveforms representative of impedance components within an IPG signal may be expressed mathematically as follows. Extracted waveforms may be time-dependent, where I(t) describes a resistive component of the impedance, Q(t) represents a reactive component and |Z(t)| represents the overall magnitude component of the impedance, where all three are measured in the units of Ohms. φ(t), the phase, is representative of a relationship between the resistive and reactive components of the signal I=real(\vec{Z}), Q=imag(\vec{Z}), where \vec{Z} is the impedance of the tissue. A different representation of the impedance may be given by |Z|=abs(\vec{Z}), φ=tan⁻¹(Q/I). Further details on mathematical representations of an IPG signal are provided below.

Waveforms may also be extracted at differing time scales, for instance to filter out either high or low frequency variations, or to focus on elements of the IPG signal having higher or lower amplitudes. Thus, the change in impedance components of a waveform may be examined on time scales on the order of fractions of seconds, seconds, minutes, and several hours. The change in impedance components of the waveform may also be examined on differing amplitude scales. For example, an impedance waveform associated with the cardiac cycle may show variation on relatively short time scales, on the order of fractions of seconds, and may show magnitude changes in an impedance amplitude waveform on the order of hundredths to tenths of an Ohm and thousandths to hundredths of a degree in an impedance phase waveform. In contrast, a baseline impedance waveform, associated with slow adjustments in cerebral blood volume, may demonstrate variations on longer time scales, such as on the order of several minutes or hours, and may be represented by a magnitude of tens to hundreds of ohms in an impedance amplitude waveform and 0-90 degrees in the impedance phase waveform.

According to some embodiments of the present disclosure, the at least one processor may be configured to determine at least one characteristic of an extracted impedance waveform. As used herein, a characteristic of a waveform is a quantity or value characterized by at least one measure of a waveform, and may be related to either or both of an amplitude or temporal feature. For example, the amplitude of an identifiable feature, such as a peak, of a waveform may be a waveform characteristic. In another example, the timing distance between peaks in separate cardiac cycles may be waveform characteristics.

Some waveform characteristics may be related to an amplitude measure. For example, waveform characteristics may be determined in any waveform extracted from an IPG signal, including, for example, an impedance magnitude waveform, an impedance phase waveform, an impedance resistance waveform, and an impedance reactance waveform. Waveform characteristics may be determined within a repeating cycle in an impedance waveform. For example, an impedance magnitude waveform displays a repetitious pattern of spikes. Each spike corresponds to an individual cardiac cycle of a subject and may be treated as a separate data set. Thus, identifying a waveform characteristic within an impedance magnitude waveform may include identifying the same characteristic, such as the height of a peak, in each spike corresponding to an individual cardiac cycle. Waveform characteristics may also be determined in waveforms corresponding to the respiratory cycle or ICP slow-wave variations. ICP slow-wave variations may be associated with the body's autoregulation cycle. Waveform characteristics may also be determined in by comparing features between multiple extracted waveforms. Furthermore, as will be described in more detail below, waveform characteristics may be determined from supplemental waveforms, extracted, for example, from additional IPG signals, blood pressure signals, an ECG signal, or a CO2 concentration signal. For example, the peak to peak amplitude value of a blood pressure signal may be an waveform characteristic. Determined waveform characteristics may be used to estimate intracranial physiological parameters.

Some waveform characteristics may be related to a temporal measure. For example, the elapsed time between two identifiable features, such as peaks, of a waveform may constitute a temporal characteristic. Temporal characteristics may be determined in any waveform extracted from an IPG signal, including, for example, an impedance magnitude waveform, an impedance phase waveform, an impedance resistance waveform, and an impedance reactivity waveform. Temporal characteristics may be determined within a repeating cycle within an impedance waveform. Identifying a temporal characteristic within an impedance magnitude waveform may include identifying the same characteristic, such as the time interval between a first peak and a second peak, in each spike corresponding to an individual cardiac cycle. Temporal characteristics may also be determined in waveforms corresponding to the respiratory cycle or ICP slow-wave variations. Temporal characteristics may also be determined by comparing features between multiple extracted waveforms. Furthermore, as will be described in more detail below, temporal characteristics may be determined from supplemental waveforms, extracted, for example, from additional IPG signals, blood pressure signals, an ECG signal, and CO2 concentration signals. For example, the elapsed time between an R-wave peak of an ECG signal and an identifiable peak of an impedance magnitude waveform may constitute a temporal characteristic. Determined temporal characteristics may be used to estimate intracranial physiological parameters.

Exemplary embodiments consistent with the present disclosure may include estimating ICP based on at least one characteristic of an IPG waveform. In some exemplary embodiments, ICP estimation may be performed based on at least one IPG waveform characteristic and at least one other characteristic extracted from a supplemental waveform, for example an arterial blood pressure waveform or an autoregulation waveform.

In an impedance waveform extracted from an IPG signal, information about the subject's body may be contained in both amplitude and temporal characteristics of the impedance components of the waveform. Information about the subject's body may also be contained in a comparison between amplitude and temporal characteristics of the waveform, or in a comparison between characteristics of an impedance waveform with characteristics of a supplemental waveform, extracted, for instance, from another IPG signal, a blood pressure signal, an electrocardiogram signal, or a CO2 concentration signal.

Information about the subject's body contained in extracted impedance waveforms may be indicative, for example, of intracranial physiological parameters within a subject's brain. Hemodynamic parameters may include, for example, intracranial pressure, cerebral blood volume, cerebral blood flow, cerebral perfusion pressure, and any other parameter that might be at least partially reflective of cerebral conditions.

An IPG signal associated with a subject's brain may be obtained from the left or right hemisphere of a subject's brain, and may also include a signal obtained from a global cranial measurement receiving information from both hemispheres at once. An IPG signal obtained from one hemisphere of a subject's brain may be indicative of hemodynamic characteristics in the hemisphere from which it is obtained, or hemodynamic characteristics from the opposing hemisphere.

Processor 160 may be configured to receive a signal from one or more electrodes 110, included in exemplary headset 120 of FIG. 1. Electrodes 110, may be arranged singly, in pairs, or in other appropriate groupings, depending on implementation. The electrodes on exemplary headset 120 may be arranged so as to obtain IPG signals. IPG signals may be measured by two sensor sections 150, disposed on the right and left sides of the head to correspond with the right and left hemispheres of the brain, for example. While only one sensor section 150 is shown in FIG. 1, an opposite side of the subject's head might include a similar electrode arrangement. Each sensor section 150 may include one pair of front electrodes, front current electrode 111 and front voltage electrode 112, and one pair of rear electrodes, rear current electrode 114, and rear voltage electrode 113. The distance between the pairs may be adjusted such that a particular aspect of an intracranial physiological condition is satisfied. The electrode configuration depicted in FIG. 1 is only one example of a suitable electrode configuration. Additional embodiments may include more or fewer electrodes 110, additionally or alternatively arranged in different areas of exemplary headset 120. Other embodiments may include electrodes 110 configured on an alternatively shaped headset to reach different areas of the subject's head as compared to the exemplary headset 120.

Pairs of electrodes 110 may include a current output electrode and a voltage input electrode. For instance, front current electrode 111 and front voltage electrode 112 may form an electrode pair. In one embodiment, an output current may be generated by cerebral perfusion monitor 130 and passed between front current electrode 111 and rear current electrode 114. The output current may include an alternating current (AC) signal of constant amplitude and stable frequency in the range of 1 KHz to 1 MHz. An input voltage induced on the head due to the output current may be measured between front voltage electrode 112 and rear voltage electrode 113. An input voltage may be measured at the same frequency as the output current. A comparison between the output current signal, e.g. a measurement signal, and the input voltage signal, e.g. a response signal, may be used to extract an impedance waveform of the subject. More specifically, a magnitude of the bioimpedance may be computed as a ratio of the input voltage signal amplitude to the output current amplitude signal, and a phase of the bioimpedance may be computed as the phase difference by which the output current signal leads the input voltage signal. Additional impedance components may be computed from the output current signal and the input voltage signal, or from the bioimpedance magnitude and phase, as required.

An IPG signal may also include output current at more than a single AC frequency. The output current may include a set of predefined frequencies and amplitudes, for example in the range of 1 KHz to 1 MHz, with detection of the measured voltage at all of the frequencies or a part of the frequency range.

Blood and fluid flow into and out of the head, and more specifically, the brain, may result in changes in the cranial bioimpedance characterized by the IPG signal measured by electrodes 110. Bioimpedance changes may correlate with blood content and blood pressure in the head and brain, as well as the contents and pressure of other fluids within the brain. The cardiac cycle, respiration cycle, and ICP slow-waves cycle affect the content and pressure of both blood and other fluids in the brain. In general, because blood and other fluids have a relatively low impedance when compared with tissue found in the head, higher blood or fluid content results in a lower impedance magnitude. Impedance changes associated with differing blood and fluid content and pressure within the brain may also cause variations in the frequency response of the brain impedance. Comparing bioimpedance measurements at different frequencies may provide additional information indicative of hemodynamic characteristics.

The exemplary headset 120 may include further devices or elements for augmenting bioimpedance measurements or for performing measurements in addition to bioimpedance measurements, such as an additional sensor or sensors 140. In one embodiment, additional sensor 140 may include, for example, a light emitting diode 141 and a photo detector 142 for performing Photo Plethysmography (PPG) measurements either in conjunction with or as an alternative to bioimpedance signal measurements. The exemplary headset 120 may further include various circuitry 170 for signal processing or other applications and may include the capability to transmit data wirelessly to cerebral perfusion monitor 130 or to other locations. In an additional embodiment, cerebral perfusion monitor 130 may be integrated with headset 120. Although illustrated in the example of FIG. 1, additional sensor 140 and circuitry 170 may be omitted.

Exemplary headset 120 may include various means for connecting, encompassing, and affixing electrodes 110 to a patient's head. For example, headset 120 may include two or more separate sections that are connected to form a loop or a band that circumscribes the patient's head. Any of these aspects, including bands, fasteners, electrode holders, wiring, hook-and-loop connector strips, buckles, buttons, clasps, etc. may be adjustable in order to fit a patient's head. Portions of exemplary headset 120 may be substantially flexible and portions of the exemplary headset 120 may be substantially inflexible. For example, electrode-including portions of exemplary apparatus 120 may be substantially inflexible in order to, among other things, substantially fix electrodes 110 in specific anatomical positions on the patient's head. In addition to or in the alternative, other portions, such as bands or connectors holding the exemplary headset 120 to a patient's head, may be substantially flexible, elastic and/or form fitting.

Any portion of exemplary headset 120 may be specifically designed, shaped or crafted to fit a specific or particular portion of the patient's anatomy. For example, portions of exemplary headset 120 may be crafted to fit near, around or adjacent to the patient's ear. Portions of exemplary headset 120 may be specifically designed, shaped or crafted to fit the temples, forehead and/or to position electrodes 110 in specific anatomical or other positions. Portions of the exemplary headset 120 may be shaped such that electrodes 110 (or other included measurement devices) occur in specific positions for detecting characteristics of blood and fluid flow in the head or brain of the patient. Examples of such blood flow may occur in any of the blood vessels discussed herein, such as the arteries and vasculature providing blood to the head and/or brain, regardless of whether the vessels are in the brain or feed the brain.

Exemplary headset 120 may include features suitable for improving comfort of the patient and/or adherence to the patient. For example exemplary headset 120 may include holes in the device that allow ventilation for the patient's skin. Exemplary headset 120 may further include padding, cushions, stabilizers, fur, foam felt, or any other material for increasing patient comfort.

As mentioned previously, exemplary headset 120 may include one or more additional sensors 140 in addition to or as an alternative to electrical or electrode including devices for measuring bioimpedance. For example, additional sensor 140 may include one or more components configured to obtain PPG data from an area of the patient. Additional sensors 140 may comprise any other suitable devices, and are not limited to the single sensor illustrated in FIG. 1. Other examples of additional sensor 140 include devices for measuring local temperature (e.g., thermocouples, thermometers, etc.) and/or devices for performing other biomeasurements.

Exemplary headset 120 may include any suitable form of communicative mechanism or apparatus. For example, headset 120 may be configured to communicate or receive data, instructions, signals or other information wirelessly to another device, analytical apparatus and/or computer. Suitable wireless communication methods may include radiofrequency, microwave, and optical communication, and may include standard protocols such as Bluetooth, WiFi, etc. In addition to, or as an alternative to these configurations, exemplary headset 120 may further include wires, connectors or other conduits configured to communicate or receive data, instructions, signals or other information to another device, analytical apparatus and/or computer. Exemplary headset 120 may further include any suitable type of connector or connective capability. Such suitable types of connectors or connective capabilities may include any standard computer connection (e.g., universal serial bus connection, firewire connection, Ethernet or any other connection that permits data transmission). Such suitable types of connectors or connective capabilities may further or alternatively include specialized ports or connectors configured for the exemplary apparatus 100 or configured for other devices and applications.

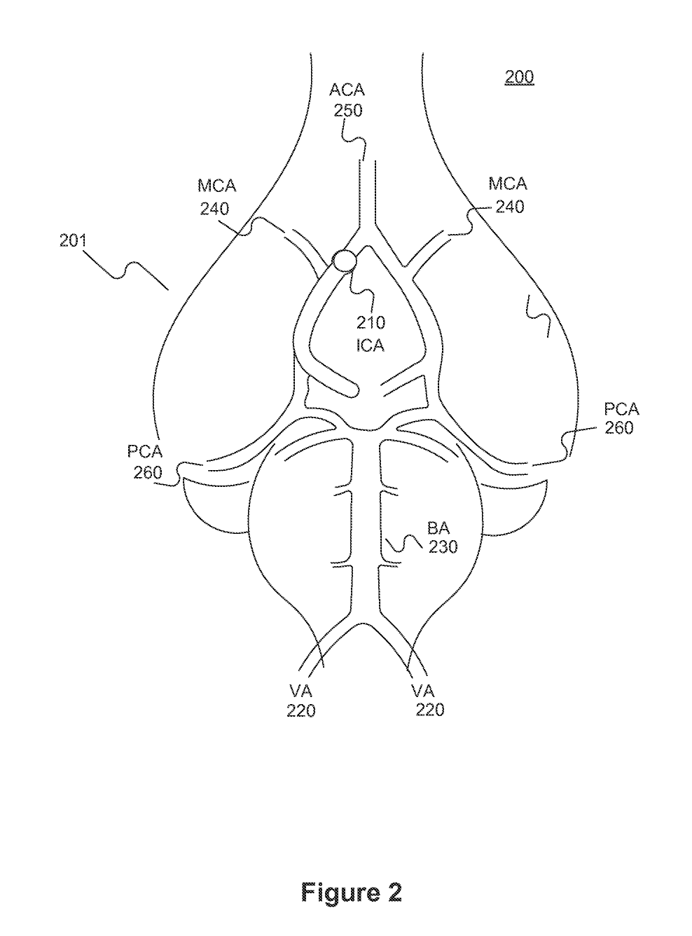

FIG. 2 provides a diagrammatic representation of major features of the cerebral vasculature 200. The cerebral vasculature in FIG. 2 is viewed from below the brain, with the top of the page representing the front of a subject. The blood supply to the brain 201 comes from four main arteries traversing the neck. The larger two are the right and left internal carotid arteries (ICA) 210, in the front part of the neck. The vertebral arteries (VA) 220 are located in the back of the neck and join to form the basilar artery (BA) 230. The internal carotid arteries and the basilar arteries are connected by Posterior Communicating Artery (not shown) and Anterior Communicating Artery (not shown) to form the Circle of Willis (COW). In an ideal patient, the COW is a network of connected arteries that allows blood supply to the brain 201 even when one or more of the feeding arteries is blocked.

The main arteries that supply blood to the brain 201 are the Middle Cerebral Arteries (MCAs) 240, Anterior Cerebral Arteries (ACAs) 250, and Posterior Cerebral Arteries (PCAs) 260.

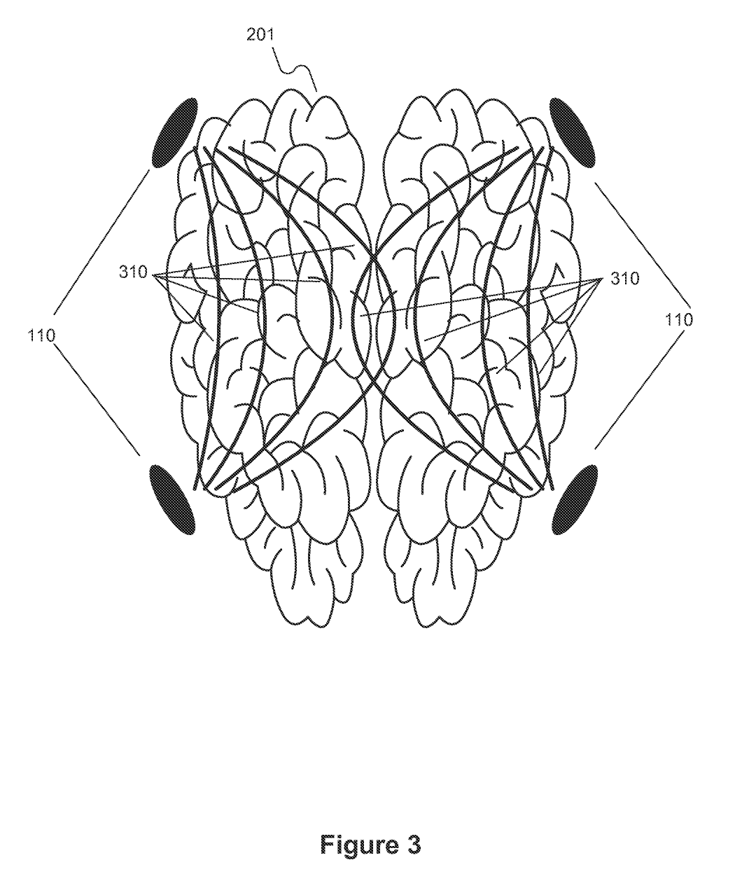

FIG. 3 provides a diagrammatic representation of exemplary impedance signal pathways 310 in the brain 201 of a subject. The exemplary configuration illustrates multiple signal pathways 310 through each of the right and left brain hemispheres. The multiple signal pathways extend between electrodes 110 affixed to the head of a subject via headset 120. The impedance of the signal pathways 310 may be influenced by the presence or absence of blood along the pathway, because blood has a relatively low impedance. At least some of the signal pathways 310 may be coincident with brain vasculature. Signal properties may thus be measured that are indicative of hemodynamic characteristics, such as pressure, blood flow, or volume, in the blood vessels of the brain 201. Changes in bioimpedance may thus be indicative of changes in pressure, blood flow, or blood volume, in the brain 201. Signal pathways 310 depicted in FIG. 3 are representative of only a small number of an infinite number of pathways which may exist in the general area of signal pathways 310.

In some embodiments consistent with the present disclosure, the at least one IPG signal associated with the brain of the subject may include at least a left hemisphere IPG signal and a right hemisphere IPG signal. A left or right hemisphere IPG signal, as used herein, may include an IPG signal reflective of impedance characteristics of the side of the brain with which it is associated. Left and right hemisphere IPG signals may be obtained from either side of the head, as impedance characteristics of the left hemisphere may be obtained from a location on the right side of a subject's head, and vice versa. An IPG signal relating to a particular side of a subject's brain may also be obtained from other locations, such as on the neck of a subject, where, for example, carotid arteries are located.

An IPG signal may also be obtained through rearrangement of the voltage and current electrode pairs. For example, a frontal pair of voltage and current electrodes may be used to provide a frontal IPG signal and a rear pair of voltage and current electrodes may be used to provide an intracranial IPG signal. The left/right arrangement and frontal/intracranial arrangements may be electronically or mechanically switched using processor 160. To obtain more than one IPG measurement, for example by measuring simultaneously both right and left IPG signals, an alternating current frequency used in each of the measurements may be different, to differentiate between the sides. Using this technique, the voltage signal obtained from each side may be demodulated with respect to the corresponding current or with respect to the current delivered in the opposite side.

According to embodiments consistent with the present disclosure, the IPG waveforms may be utilized to determine ICP, and, more specifically, mean ICP. As noted above, the ICP may be influenced by three general bodily factors: CBV, edema status, and CSF volumes. The ICP may also be influenced by several cyclical parameters of the body, including but not limited to, the cardiac cycle, the respiration cycle, and the ICP slow-wave cycle corresponding to the body's natural vascular autoregulation of cerebral blood flow. These three factors may affect the ICP at different time scales. The highest frequency variations in the ICP signal may be associated with the cardiac cycle and the arterial blood pressure changes induced by the heart's beating. At lower frequencies, the influence of the respiration cycle and corresponding changes to intrathoracic pressure may be detected in the ICP. At even lower frequencies, ICP slow-waves or plateau-waves with periods in the order of tens of seconds to several minutes correspond to the reactivity time scale of the vascular autoregulation mechanism. ICP slow-waves are pressure variations having a period of between approximately twenty seconds and several minutes. ICP slow-waves may be associated with physiological cerebral changes caused by the vascular autoregulation mechanism.

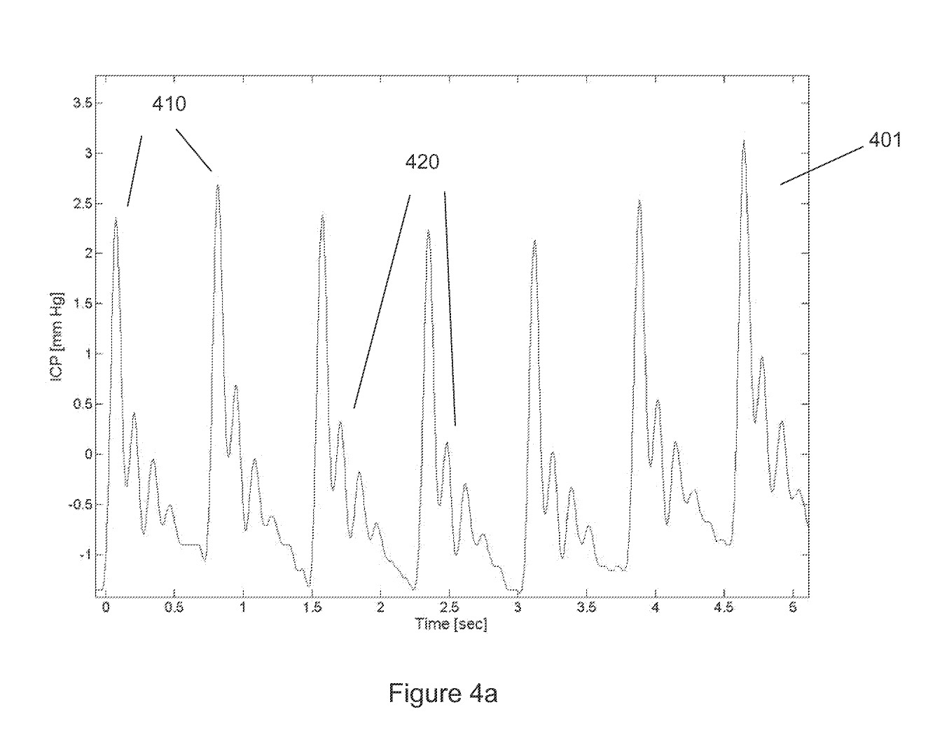

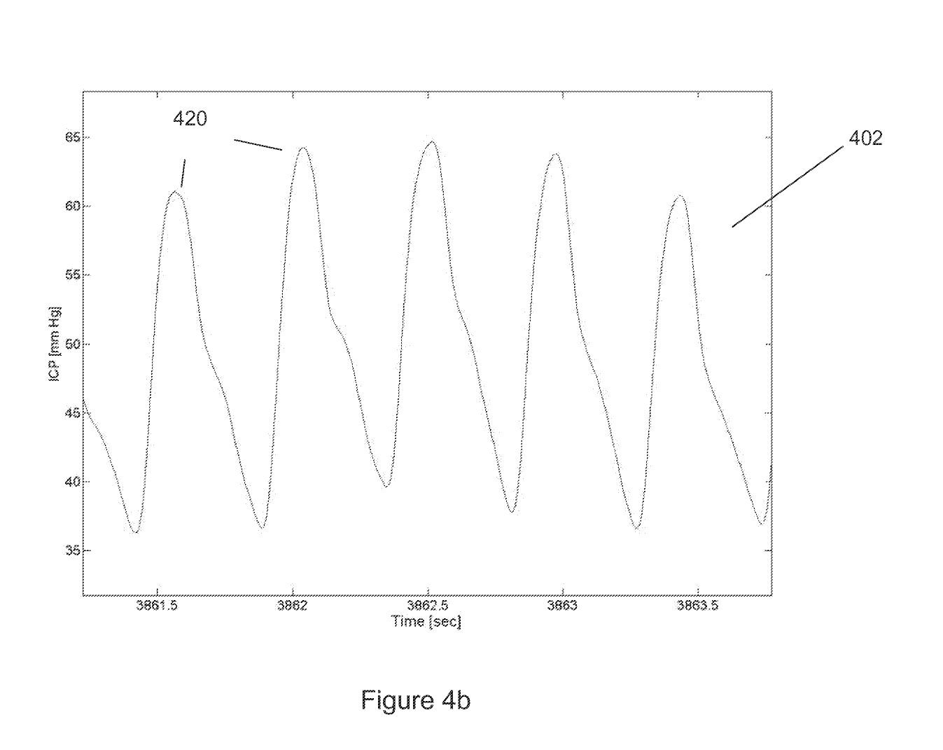

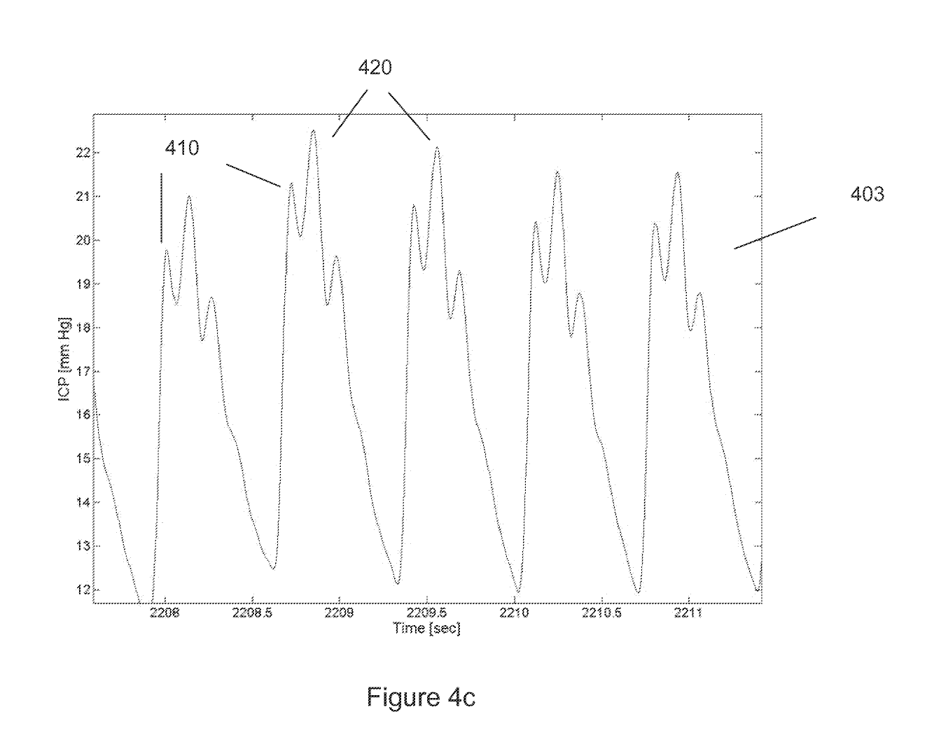

FIGS. 4a-4c illustrate ICP waveforms obtained through conventional, invasive measures. ICP waveform 401, illustrated in FIG. 4a provides a diagrammatic representation of an ICP waveform obtained from a healthy brain under normal conditions, with an ICP ranging between −1 and 2.5 mm Hg. The first peak (P1) 410 is significantly higher than the second peak (P2) 420 in this waveform. In addition, the signal waveform is characterized by high roughness. ICP waveform 402, illustrated in FIG. 4b provides a diagrammatic representation of an ICP waveform obtained from a pathological brain, with an ICP ranging between 35 and 60 mm Hg. In ICP waveform 402, P1 410 is not seen, because it is screened by P2 420 which is much higher. In addition, the roughness of the signal waveform is very low—it has only a few characteristic features. ICP waveform 403, illustrated in FIG. 4c provides a diagrammatic representation of an ICP waveform obtained from a brain under elevated ICP conditions, with the ICP ranging between 12 and 21 mm Hg. In this figure, P2 420 is slightly higher than P1 410, and the roughness is still high.

Characteristics that are evident in these ICP waveforms vary depending on the condition of the subject's brain. For example, the ratio of a first peak (P1) 410 to a second peak (P2) 420 varies between the signals. In the healthy brain, P1 410 is significantly higher than P2 420. In the pathological brain, P2 420 is expanded in height and width to the point where it screens and obscures P1 410. Finally, in the elevated ICP brain, P1 410 is lower than P2 420. Thus, the ratio of P1 to P2 is an indicator that may correlate with the mean value of the ICP. As another example evident in these waveforms, the roughness of each ICP waveform decreases with an increasing mean ICP. The roughness of a waveform measures the frequency of identifiable variations within the waveform. The P1 to P2 ratio and roughness of the ICP waveforms, as illustrated in FIGS. 4a-c, are exemplary identifiable characteristics in an ICP waveform.

The concavity of the cardiac complex, which may be defined as the relation between the time period the signal is above a certain threshold (e.g. the average of the minimal and maximal value) and the duration of the complex (which equals one divided by the heart rate), may also be indicative of the mean value of ICP. In the healthy brain the concavity ratio is small, as can be seen in FIG. 4a, while in the pathological brain the concavity ratio is larger, as can be seen in FIG. 4b. The concavity ratio is a clinical parameter which may correlate with the mean value of ICP.

Peak to peak (P2P) measurements may also be indicative of a mean value of the ICP. For each cardiac complex in the ICP waveform, the peak to peak measure may be defined as the difference between the maximal value and the minimal value. The cardiac complexes in the ICP signal correspond to the volume of blood entering into the brain each beat, which are defined as Cerebral Stroke Volume (CSV). CSV and Cerebral Blood Flow (CBF) are interlinked, as CBF equals the sum of CSV's over a period of one minute. The peak to peak measure of the cardiac complexes in the ICP signal, thus, may also correlate well with the mean value of ICP. The foregoing represent only exemplary characteristics that may be identified within ICP signals that may be indicative of mean ICP value.

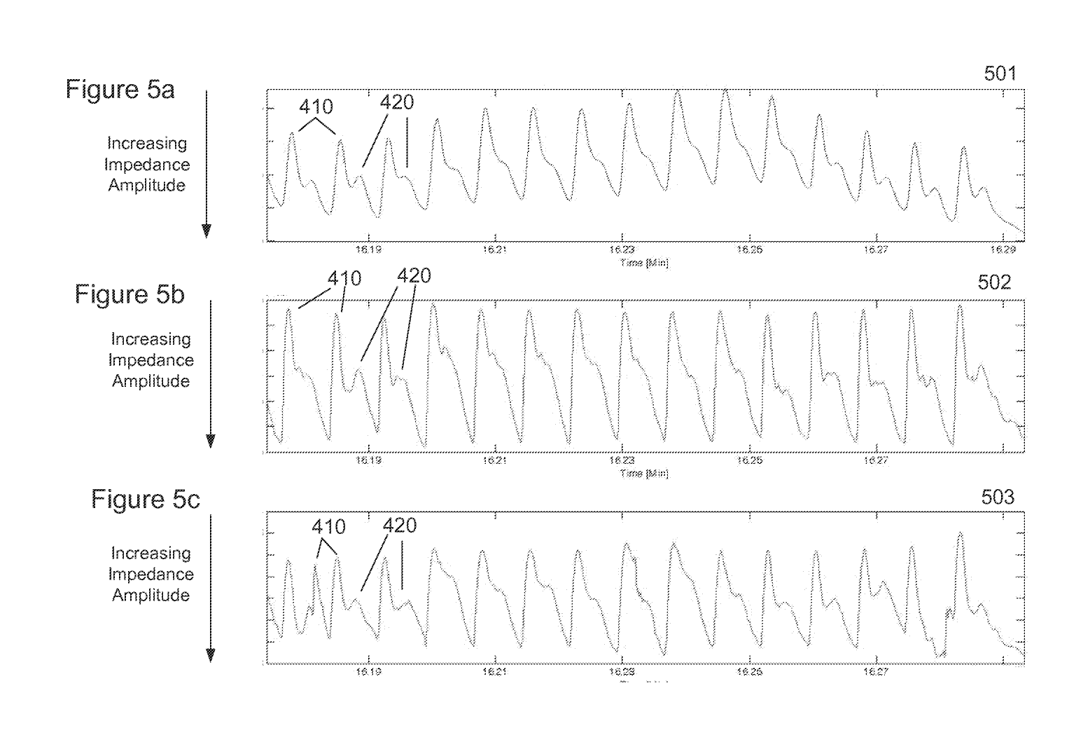

According to embodiments consistent with the present disclosure, at least one intracranial physiological parameter, including intracranial pressure, may be estimated from at least one impedance waveform or characteristic extracted from an IPG signal. FIGS. 5a-c illustrate an ICP signal recorded simultaneously with an IPG signal. FIG. 5a illustrates the ICP signal 501, while FIGS. 5b and 5c respectively illustrate an impedance magnitude waveform 502 extracted from the IPG signal and a phase waveform 503 extracted from the IPG signal. Each of these signals is illustrated over a time period corresponding with a single respiration cycle.

In FIGS. 5a-c, the impedance magnitude waveform 502 and the phase waveform 503 demonstrate characteristics that correlate with characteristics within the ICP signal 501. FIG. 5a provides a diagrammatic representation of an exemplary ICP signal 501. FIG. 5b provides a diagrammatic representation of an exemplary impedance magnitude waveform 502, recorded simultaneously to the ICP signal 501. FIG. 5c provides a diagrammatic representation of an exemplary impedance phase waveform 503, recorded simultaneously to the ICP signal 501.

For example, all three signals demonstrate P1 410 and P2 420 characteristics. A rise and fall of the mean ICP associated with a respiration cycle can also be seen in the ICP signal 501. Coinciding with the rise and fall of the mean ICP is a similar rise and fall in the height of P2 420 within that signal. Impedance magnitude waveform 502 and impedance phase waveform 503 also demonstrate a rise and fall in the height of P2 420 that coincides with the rise and fall of the mean ICP as shown in ICP signal waveform 501. Thus, information about the mean ICP may be obtained, for instance, from variations in the height of P2 420 within an impedance magnitude waveform 502 or an impedance phase waveform 503. These characteristics are detailed here for exemplary purposes only, as they are readily discernible from mere observation of waveforms 501, 502, and 503. Through additional analysis techniques, as will be discussed in more detail below, additional characteristics may be identified within impedance magnitude waveform 502 or impedance phase waveform 503.

It can be seen from FIGS. 5a-5c, that the IPG waveform closely follows the changes in the ICP waveform, and shows strong similarity to the ICP waveform. Both IPG amplitude and phase waveforms show strong correlations with ICP changes.

A measured IPG waveform may show changes due to relative changes in the blood volume of the tissue through which the IPG current flows and due to additional hemodynamic parameters. The blood volume may vary according to the instantaneous blood pressure and flow during a cardiac cycle, and this change may be captured by the IPG waveform in a cardiac cycle. In clinical testing, dynamic components of IPG waveforms correlate well with dynamic components of ICP waveforms. However, because IPG waveforms measure relative changes in tissue blood volume, mechanical brain pulsation, and CSF pulsatility, additional analysis of the dynamic components of the IPG waveform may be necessary in order to determine, with the assistance of physiological calibration, static, or mean values of ICP.

The dynamic components of ICP waveforms, and their measured IPG analogs, may also be classified by their spectral properties. The highest frequency signal, with the fastest pulsatility, results from the cardiac complexes. Every heart beat drives blood flow to the brain, affecting the measured ICP. At lower frequencies, the signal may be modulated by respiration. Breathing in and out alters the pressure on the jugular veins, which, in turn, alters the pressure required for blood to flow out of the brain, affecting the measured ICP. At still lower frequencies, there are slow waves which correspond to the reactivity time scale of the vascular autoregulation (AR) mechanism. The body naturally adjusts blood flow characteristics, through mechanisms such as vasodilation and vasoconstriction; such changes may take tens of seconds up to tens of minutes to be affected.

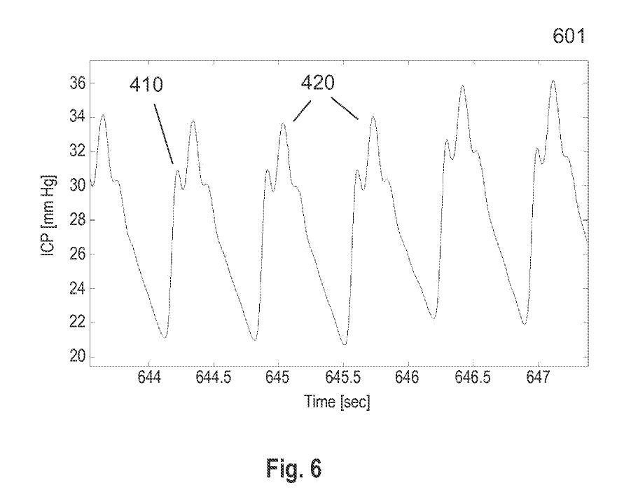

In some embodiments consistent with the present disclosure, estimating a mean ICP may include eliminating or normalizing dynamic components of an ICP waveform or its representative IPG waveform. After adjusting for the relative amplitudes of pulsatile features of the ICP waveform that correspond to the cardiac complexes, respiratory cycle, and autoregulation mechanism, the mean value of the ICP remains. From the adjustments necessary to determine a mean ICP value based on an ICP waveform, the adjustments necessary to determine a mean ICP value based on an IPG signal corresponding to an ICP waveform may be determined. All of the factors described above may be useful in monitoring the cranial condition of a patient. FIGS. 6-8, as discussed below, provide additional illustrations of the effects of some of the above-discussed physiological factors on ICP.

FIG. 6 illustrates an ICP waveform 601 of a brain with a high level of edema, or fluid buildup. In the illustrated ICP waveform, the height of P2 420 shows a significant increase with respect to the expected level in a healthy brain. Thus, the height of P2 420 may be an indicator of edema level in the brain. As described above, edema level is a contributing factor to ICP elevation, and thus, increased P2 420 height may be indicative of increased ICP mean value in the brain.

In some embodiments consistent with the present disclosure, a working position on a brain compliance curve may be estimated based on an extracted waveform. As described above, determining a mean ICP may require normalizing for or adjusting for the relative amplitudes of pulsatile features in an ICP or representative IPG waveform. A correlation between the relative measures of the ICP waveform (or representative IPG waveform) and the mean value of the ICP waveform may be determined through an understanding of a compliance curve of the brain. The compliance curve of the brain may be understood as the relationship between brain volume and pressure.

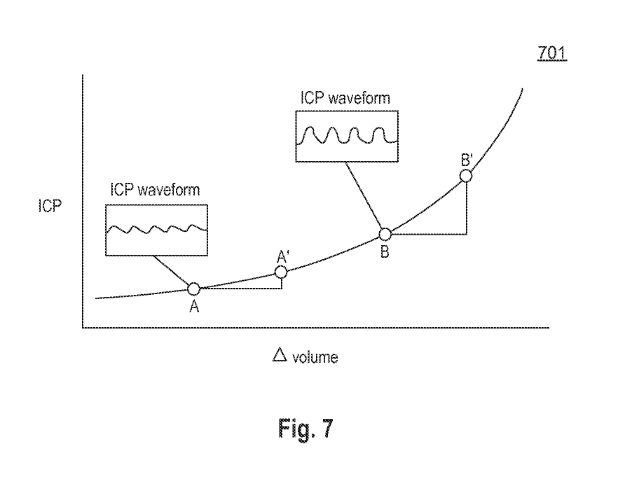

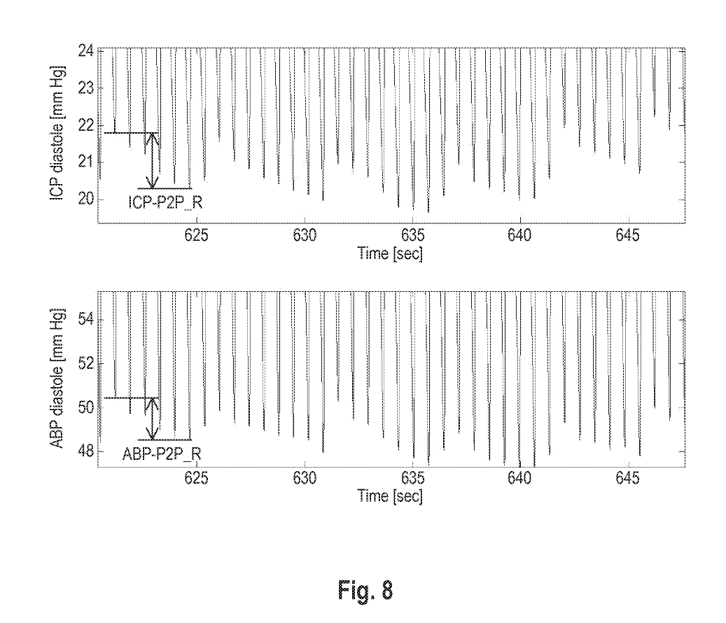

FIG. 7 illustrates a brain compliance curve 701. Brain volume includes brain tissue volume, Cerebral Blood Volume (CBV) and Cerebral Spinal Fluid (CSF). Changes in the brain volume may be driven primarily by changes in CBV and CSF. As FIG. 7 illustrates, as the brain volume (x-axis) increases, smaller changes in the brain volume correlate with increasingly larger changes in ICP. Thus, as long as CSV and CSF do not fluctuate too greatly between successive cardiac complexes, the size of variations in the peak to peak measure of the ICP waveform may be indicative of a working position on a brain compliance curve 701, which may further correlate with a mean value of the ICP. For example, a high peak to peak measure of ICP may be indicative of a high CBV (corresponding to B-B′ in FIG. 7), while a low peak to peak measure of ICP may be indicative of a low CBV (corresponding to A-A′ in FIG. 7). This can also be seen in FIGS. 4a and 4b, where the ICP peak to peak measures are 3.5 mm, and 24 mm Hg, respectively. The peak to peak measure of ICP, therefore, may be an indicator of the mean value of ICP.

Furthermore, the peak to peak measure of an ICP waveform during a single heartbeat complex may also be an indicator of CSF maintenance functioning. As described above, CSF volume maintenance is among the factors that determine mean ICP. In some cases, doctors perform CSF maintenance on patients. However, when CSF is not artificially maintained by physicians, the peak to peak measure of the ICP waveform may be indicative of CSF maintenance functioning. In a situation where CSF fails to flow out of the brain, either due to low CSF availability or blockage of CSF flow, the effect of variations in blood flow on the ICP waveform is larger, as a brain that retains CSF will have a relatively large brain volume, and thus be further to the right on the compliance curve.

In some embodiments consistent with the present disclosure, waveform characteristics extracted from an impedance waveform associated with a patient respiratory cycle may be utilized for estimating a working position on a brain compliance curve. Characteristics of the ICP waveform associated with the respiratory cycle may also be valuable in determining a mean value of ICP. Respiration results in changes to the intrathoracic pressure. Inhalation increases the intrathoracic pressure, thus increasing the external pressure on the jugular vein, which in turn decreases blood outflow from the brain, thereby increasing CBV and hence ICP. ICP measurements taken during a Valsalva maneuver illustrate this. In the Valsalva maneuver, patients may increase their intrathoracic pressure by attempting to expire against a closed airway. During a Valsalva maneuver, measured ICP may increase to values of above 30 mm Hg due to the increase in CBV.

FIG. 8 illustrates diastolic values of ICP and ABP during respiratory cycles. In FIG. 8, the effects of respiratory modulation can be seen in a comparison between ICP and arterial blood pressure (ABP) waveforms. Each downward spike on the graphs shown is the ICP or ABP measure at a diastole portion of the cardiac cycle. As shown, the minimum ICP and ABP show a cyclical pattern over the course of a respiration cycle. The minimum ICP and ABP reach their lowest points during an exhalation phase of a respiratory cycle. As illustrated in FIG. 8, respiratory modulations of the respiratory peak-to-peak measures of ICP and ABP (ICP-P2P_R and ABP-P2P_R, respectively) equal 1.5 mm and 2 mm respectively.

As discussed above, measuring the brain's working position on the compliance curve through ICP may be facilitated by a steady CSV. In some patients, however, the CSV between successive cardiac cycles may not be steady enough to allow for an accurate measurement of the compliance curve working position through ICP. Because the respiratory cycle affects ICP independently of CSV, it may provide a supplemental measure indicative of a brain's position on the compliance curve. ABP, which may be conveniently measured, may be used to provide this supplemental measure. Because ICP is contributed to by factors related to blood flow (CBV) as well as factors not related to blood flow (e.g. CSF level and edema level), a comparison between ICP and ABP may help serve to separate these influences. The difference between changes in blood pressure over a respiratory cycle and changes in ICP over the same respiratory cycle may therefore be indicative of the working position of the brain on the compliance curve. This may be described mathematically as follows. Define CC-R=(ICP-P2P-R)−(ABP-P2P-R). CC-R indicates the working location of the brain on the compliance curve. Thus, subtracting the respiratory peak-to-peak measure of arterial blood pressure from the respiratory peak-to-peak measure of intracranial pressure results in a measure indicative of a working position of the brain on the compliance curve.

Additionally, the ratio between a peak to peak ICP measurement during a heartbeat complex at peak inspiration and at peak expiration may be utilized to indicate the current compliance curve working location, through calibration with the ABP signal.

In some embodiments consistent with the present disclosure, characteristics of the ICP waveform associated with an autoregulation, or slow wave, cycle may be used to determine a mean value of ICP. The pressure reactivity index (PRX), for example, is a measure correlated with the mechanical functioning of the autoregulation mechanism, and may thus be correlated with a mean value of ICP.

As discussed above, extracted waveforms representative of impedance components within an IPG signal may be expressed mathematically in an {I,Q} (e.g. in-phase, quadrature) representation. In-Phase (I) and quadrature (Q) signals representative of voltage (v) and current (i) may be extracted from a recorded impedance signal. Such extraction may yield Ic, Qc, Iv, and Qv. A complex impedance waveform \vec{Z} may be computed from the extracted current and voltage waveforms as follows. \vec{Z}=(Iv+j Qv)/[(Ic+j Qc)/R0], where j=\sqrt{−1}, and \vec{Z}=impedance of the tissue under study (TUS).

Because \vec{Z} is a complex waveform, it may be represented using the {I,Q} (e.g. in-phase, quadrature) representation, wherein, I=real(\vec{Z}), Q=imag(\vec{Z}). An alternate representation of the impedance may be also given by the amplitude and phase measurements, |Z|=abs(\vec{Z}),

$$ϕ = {tan}^{- 1} ( \frac{Q}{I} ) .$$

Each of the waveforms are time-dependent, where I(t) describes the resistive portion of the impedance, Q(t) describes the reactance portion and |Z(t)| characterizes the overall magnitude of the impedance, where all three are measured in units of Ohms. φ(t), the phase angle signal, corresponds to the relation between the reactance and the resistance and may be measured in degrees.

In the analysis of the IPG waveform, both the high pulsatility components, for example, the heart complexes and the respiratory modulation, and low pulsatility components, for example, auto regulation slow-waves and edema development, can be seen in all four measures: I(t), Q(t), |Z(t)|, φ(t).

The waveform of the IPG signal may be processed with various techniques, such as spectral analysis and mode decomposition techniques to analyze the waveform at varying time scales. For example, waveforms associated with differing physiological processes, such as the cardiac cycle, respiration cycle, or slow-wave cycle, may be extracted from the IPG signal using mode decomposition techniques to eliminate signal elements that occur at frequencies not associated with the appropriate physiological process. The waveform may then be analyzed with respect to the above described pathological indicators and be used to extract the mean value of the ICP and the waveform complex noninvasively. Waveforms for analysis may similarly be extracted from other types of signals, such as ABP signals and ECG signals.

The indicators described above with respect to measuring a mean ICP value, e.g. P1/P2 relation, roughness, concavity measure, P2P measures, CSF functioning, edema indications, and autoregulation status may be measured or determined using each of the IPG waveforms: I(t), Q(t), |Z(t)|, φ(t) and/or characteristics extracted from these waveforms.

In addition to the I/Q and amplitude/phase analysis methods, any suitable mathematical handling of the data prior to extraction of parameters may be utilized. That is, a signal S such that S=function(I, Q, amplitude, phase) may be used, where the function may include mathematical manipulation based on static parameters or based on adaptive parameters which are computed according to the data. Thus, the mathematical manipulation methods may be altered according to the recorded data.

Embodiments of the present disclosure may provide for additional means of measuring hemodynamic parameters as well as means for measuring additional hemodynamic parameters. For example, in some embodiments consistent with the present disclosure, cardiac stroke volume (CSV) may be measured from IPG data. Changes in the absolute value of the impedance |Z(t)| may correspond to changes in the blood volume inside the brain. Within each cardiac complex, these changes may correspond to the CSV, the amount of blood that enters the brain every beat. This measure is also directly related to CBF, as CBF is, by definition, the sum of the CSV's over one minute.

In some embodiments consistent with the present disclosure, a mean value of ICP may be estimated from mean arterial pressure and CSV. At the frequency of the heart complexes, changes in ICP are mainly due to blood entering into the brain, and thus correlate well |Z(t)| of an IPG waveform. The amount of blood entering the brain depends on Cerebral Perfusion Pressure (CPP), which is equal to CBF times cerebrovascular resistance (CVR). Cerebrovascular resistance may be estimated from changes in the phase of the impedance waveform, as described in greater detail below. Thus, CPP may be estimated from CSV and CVR. CPP may also be correlated with ICP. That is, ICP=Mean Arterial Pressure (MAP)−CPP. Thus, by using continuous ABP data to determine mean arterial pressure, measured, for example, from a femoral artery, measuring CSV from an IPG absolute value of impedance, and measuring CVR from an IPG waveform phase, a mean value of the ICP may be estimated.

As discussed above, mean ICP may be also estimated based on an estimation of a working position on a compliance curve. In addition to methods described above, such an estimation may be assisted by estimating edema levels through analysis of impedance phase information. As described above, changes in impedance phase correlate with changes in cerebrovascular resistance. This is at least partially due to the fact that impedance phase is strongly determined by reactive components of the IPG waveform, which reflect changes in tissue structure more strongly than changes in blood flow. Thus, as the cerebral arteries experience geometric modification, e.g. expanding, contracting, stiffening, and softening, thus affecting the CVR, these changes are reflected in the phase portion of the impedance waveform.

In situations where it is only blood volume that changes from one heartbeat to the next, while blood vessels do not encounter any geometrical modifications, the phase portion of the IPG signal may be affected less significantly than the amplitude portion of the IPG signal. This may correspond to a scenario in which there is high pressure on the blood vessels from outside, corresponding to elevated ICP levels due to changes in brain tissue. In contrast, during a Valsalva maneuver, where the ICP is increased due to respiratory effects, the peak to peak measure of φ(t) in each heartbeat complex decreases with increasing ICP much more rapidly than the peak to peak of |Z(t)|. That is, comparing peak to peak measures of the phase portion of an IPG waveform during ICP increases caused by a Valsalva maneuver compared to those ICP increases caused by brain tissue changes demonstrates that the phase portion of the waveform reacts differently to ICP increases caused by brain tissue changes versus ICP increases caused by respiratory effects.

Thus, in some exemplary embodiments consistent with the present disclosure, a working position on a brain compliance curve may be estimated from phase portions of an impedance waveform associated with a respiration cycle. By measuring the peak to peak of φ(t) during a cardiac complex at peak expiration, and the peak to peak of φ(t) during a cardiac complex at peak inspiration, as well as the peak to peak values of respiratory modulation for ABP and IPG amplitude, the working location in the compliance curve may be extracted.

In some exemplary embodiments, a correlation of φ(t) and |Z(t)| may be an indicator of a mean ICP level. In healthy patients, the brain is flexible, and changes due to blood influx are accompanied with vascular geometrical changes. Thus, a timing correlation of φ(t) and |Z(t)| may be relatively low in healthy tissues with low-ICP, while, at higher levels of ICP the two signals may become more synchronized. At higher levels of ICP, when the blood vessels become stiffer due to increased pressure, any changes to the blood vessels (measured by φ(t)) due to the pulsatility of blood flow (measured by |Z(t)|) are more likely to occur with less lag between the blood flow pulse and the vessel change.

In some embodiments of the present disclosure, a plurality of impedance measurement signals at a plurality of frequencies may be utilized to generate a plurality of impedance measurements useful for estimating a physiologic parameter of a subject' brain. For example, edema levels, which may be useful for determining a working position on a brain compliance curve, as well as determining other cerebral parameters, may be estimated by measuring I(t), Q(t), |Z(t)|, φ(t) at a plurality of frequencies.

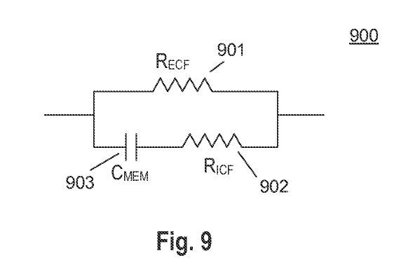

FIG. 9 illustrates a model of tissue bioimpedance. The bioimpedance of tissue may be modeled as a bioimpedance circuit 900, as illustrated in FIG. 9, as a first resistive element in parallel to a second resistive element and a capacitor. The first resistive element, R_{ECF} 901 may represent the resistance of extracellular fluid, the second resistive element, R_{ICF} 902 may represent the resistance of intracellular fluid, and the capacitor, C_{MEM} 903, may represent the capacitance of cellular membranes. When impedance is measured at a single frequency, the circuit 900 may be analyzed as a single impedance. However, changes in the frequency at which the impedance is measured change the behavior of the circuit capacitance without changing the behavior of the resistors. Thus, by analyzing impedance data at multiple frequencies, an extended picture of the value of each circuit element may be gained. The bioimpedance circuit capacitor may correspond to affects produced by cell membranes, the first resistive element may correspond to affects produced by extracellular liquid (e.g. blood flow), and the second resistive element may correspond to affects produced by intracellular liquids (e.g. edema).

Mathematically, the circuit 900 in FIG. 9 may be represented as follows, where w represents the frequency: Z(w)=R_{ECF}*[R_{ICF}/(j w C_{MEM} R_{ICF}+1)]. Measuring the tissue impedance at multiple frequencies and extracting pulsatile and non-pulsatile parameters from each of the waveforms I(t), Q(t), |Z(t)|, φ(t) at each frequency, multiple equations may be generated. Solving these equations may provide estimates of R_{ECF} 901, the resistance of extracellular fluid, R_{ICF} 902, the resistance of intracellular fluid, C_{MEM} 903, cell membrane capacitance. From these factors, the level of brain edema may be estimated. Estimates of edema may contribute to an estimate of the brain's working position on the compliance curve, as edema is among the factors that contribute to brain volume. Estimates of edema may also provide value for diagnosing other cerebral conditions.

At least one processor configured for determining edema levels may operate as follows. Using time-division multiplexing techniques, current may be delivered at frequencies ranging from 10 KHz-1 MHz over a very short time period. At each frequency, approximately 50 wavelengths of current may be delivered. Each frequency may be delivered and measured for a period of 0.5-2 milliseconds. Based on the number of frequencies delivered, and the period of measurement, the plurality of frequencies may be transmitted for 100 ms or less, 50 ms or less, 25 ms, and 5 ms or less. Because the range of frequencies are delivered and measured over time scales much shorter than typical physiological changes, the impedance measurements over multiple frequencies may be made substantially simultaneously with respect to any physiological changes, and therefore may be able to capture physiological changes.

The above described techniques using multiple frequencies may provide valuable information about additional intracranial physiological parameters beyond edema. Different components of a subject's body, e.g. blood, CSF, brain, and white matter, have different impedance spectral properties. By extracting waveform parameters from any two or more impedance signals obtained at two or more frequencies, physiological waveforms of the different cerebral components may be obtained. Additionally, by comparing the timing of events at different frequencies, for example, the time at which the systolic portion of the impedance phase reaches its maximum slope, physiological waveforms of tissues may be extracted with increased accuracy. Thus, a plurality of intracranial physiological parameters, including, for example, ICP level, edema status, autoregulation functioning, cerebral perfusion, and CSF drainage can be estimated.

Exemplary embodiments of the IPG measurement apparatus consistent with the present disclosure may include display devices, alarms, transmitters and other suitable means for conveying patient information to medical personnel. The various physiological and cerebro-hemodynamic parameters discussed herein may be measured and reported to medical personnel through a variety of means. For example, an IPG measurement apparatus may include a screen to display any parameters measured or determined. An IPG measurement apparatus may include wireless or wired network capabilities to inform medical personnel of a patient's condition via e-mail, website, or other network facilitated method.

An IPG measurement apparatus may be configured to inform medical personnel of current patient conditions, e.g. by continuously reporting mean ICP values. In some exemplary embodiments, an IPG measurement apparatus may be configured to determine and report parameter values in a simplified fashion. For example, an IPG measurement apparatus may be configured to determine and report, for example via an alarm, whether a mean ICP surpasses a certain threshold (e.g. 20 mmHg) indicating a dangerous or concerning patient condition. IPG measurement apparatus may also be configured to determine and report mean ICP values in ranges, for example by displaying a green light indicating a safe condition when ICP is below 15 mmHg, a yellow light indicating a potentially harmful or escalating condition when ICP is between 15 and 25 mmHg, and a red light indicating a dangerous condition when ICP exceeds 25 mmHg. Similarly simplified parameter determination and reporting methods may be applied to any of the parameters discussed herein.

It will be understood by a person of skill in the art that the methods presented herein for determining ICP through IPG waveform analysis are not limited to the examples presented. For example, many of the analysis methods are equally suitable for identifying features and characteristics within an ABP signal or ECG signal that may aid in the estimation of ICP, when used alone or in conjunction with data obtained from an IPG signal.

What is claimed is:

1. An intracranial physiological measurement apparatus, comprising:
at least one processor configured to:
receive at least one impedance plethysmography signal associated with a brain of a subject;
extract at least one impedance plethysmography characteristic from the impedance plethysmography signal;
and
estimate mean intracranial pressure from the at least one impedance plethysmography characteristic.

2. The apparatus of claim 1, wherein the at least one processor is further configured to:
receive an arterial blood pressure signal associated with the subject;
extract at least one arterial blood pressure characteristic from the arterial blood pressure signal; and
estimate mean intracranial pressure from the at least one impedance plethysmography characteristic and the at least one arterial blood pressure signal.

3. The apparatus of claim 1, wherein the at least one impedance plethysmography characteristic includes at least one of a peak to peak amplitude characteristic, a first peak to second peak ratio characteristic, a roughness characteristic, and a concavity characteristic.

4. The apparatus of claim 1, wherein the impedance plethysmography signal is a phase signal.

5. The apparatus of claim 1, wherein the impedance plethysmography signal is an amplitude signal.

6. The apparatus of claim 1, wherein the at least one impedance plethysmography characteristic is a correlation between a phase portion of the impedance plethysmography signal and an amplitude portion of the impedance plethysmography signal.

7. The apparatus of claim 1, wherein the at least one processor configured to estimate the mean intracranial pressure is further configured to eliminate dynamic components associated with physiological processes from the impedance plethysmography waveform.

8. The apparatus of claim 7, wherein the dynamic components include components associated with at least one of a cardiac cycle, a respiratory cycle, and an autoregulation cycle.

9. The apparatus of claim 8, wherein the at least one processor is further configured to eliminate the dynamic components based on an estimate of a working position on a brain compliance curve.

10. An intracranial physiological measurement apparatus, comprising:
at least one processor configured to:
receive at least one impedance plethysmography signal associated with a brain of a subject;
extract at least one impedance waveform associated with a physiological process from the impedance plethysmography signal; and
estimate a working position on a brain compliance curve based on the at least one impedance waveform associated with a physiological process.

11. The apparatus of claim 10, wherein the at least one impedance waveform associated with a physiological process is associated with a cardiac cycle.

12. The apparatus of claim 10, wherein the at least one impedance waveform associated with a physiological process is associated with a respiration cycle.

13. The apparatus of claim 10, wherein the at least one impedance waveform associated with a physiological process is further associated with a slow wave cycle.

14. The apparatus of claim 10, wherein the processor is further configured to:
receive at least one arterial blood pressure signal associated with the subject;
extract at least one arterial blood pressure waveform associated with a physiological process from the arterial blood pressure signal; and
estimate intracranial pressure based on the at least one impedance plethysmography waveform and the at least one arterial blood pressure waveform.

15. A cerebral hemodynamic measurement apparatus, comprising:
at least one processor configured to:
transmit and receive a plurality of impedance measurement signals at a plurality of frequencies to at least one pair of electrodes;
generate a plurality of impedance measurements of a head of a subject at the plurality of frequencies; and
estimate a physiologic parameter of a brain of the subject based on the plurality of impedance measurements.

16. The apparatus of 15, wherein the physiologic parameter is the mean value of intracranial pressure.

17. The apparatus of 15, where the physiologic parameter is a level of edema.

18. The apparatus of 15, wherein the plurality of impedance measurements include impedance phase angles.

19. The apparatus of 15, wherein the plurality of impedance measurements include absolute impedance values.

20. The apparatus of 15, wherein the plurality of impedance measurements include resistive impedance values.

21. The apparatus of 15, wherein the plurality of impedance measurements include reactance impedance values.

22. The apparatus of 15, wherein the plurality of impedance measurement signals are transmitted in less than 100 ms.

23. The apparatus of 15, wherein the plurality of impedance measurement signals are transmitted in less than 50 ms.

24. The apparatus of 15, wherein the plurality of impedance measurement signals are transmitted in less than 25 ms.

25. The apparatus of 15, wherein the plurality of impedance measurement signals are transmitted substantially simultaneously.

26. The apparatus of claim 17, wherein estimating the level of edema of the patient includes:
determining a first resistance corresponding to intracellular fluid resistance;
determining a second resistance corresponding to extracellular fluid resistance;
determining a capacitance corresponding to a cell membrane permeability.

27. The apparatus of claim 15, wherein the plurality of frequencies includes at least ten frequencies.

28. The apparatus of claim 15, wherein the plurality of frequencies ranges from 10 kHz to 1 MHz.

29. The apparatus of claim 15, wherein an impedance measurement signal at each of plurality of frequencies is transmitted for less than 2 milliseconds.
